(19) United States
(12) Plant Patent Application Publication
(10) Pub. No.: US 2006/0242742 P1
(43) Pub. Date: Oct. 26, 2006

(54) Poplar tree named 'curly poplar'

(50) POPULUS CANESCANS X ALBA; POPLAR

(76) Inventors: Richard Meilan, West Lafayette, IN (US); Samuel Grober, Evanston, IL (US)

Correspondence Address: Bose McKinney & Evans LLP, 2700 First Indiana Plaza, 135 North Pennsylvania Street, Indianapolis, IN, 46204, US

(21) Appl. No.: 11/111,700

(22) Filed: Apr. 22, 2005

(51) Int. Cl. A01H 5/00

(52) U.S. Cl. PLT/218

(57) ABSTRACT

A poplar tree particularly distinguished by having highly figured wood is disclosed.

### GENUS AND SPECIES

Populus canescans×Populus alba hybrid

### VARIETY DENOMINATION

‘Curly Poplar’

### BACKGROUND OF THE NEW PLANT

The present invention comprises a new and distinct cultivar of poplar, botanically known as Populus canescans×Populus alba hybrid, and hereinafter referred to by the cultivar name ‘Curly Poplar’. The new cultivar was found in Evanston, Ill. and propagated vegetatively by cuttings. It has been asexually reproduced by vegetative cuttings and has been shown to reproduce true to type in successive generations of asexual propagation.

### DESCRIPTION OF PHOTOGRAPHS

This new poplar tree is illustrated by the accompanying photographs which shows the tree's distinctive wood, a leaf from the tree and the overall habit of the tree. The colors shown are as true as can be reasonably obtained by conventional photographic procedures.

FIG. 1. shows the distinctive wood of the new poplar tree.

FIG. 2. shows a mature leaf from the tree.

FIG. 3. shows the overall habit of the tree.

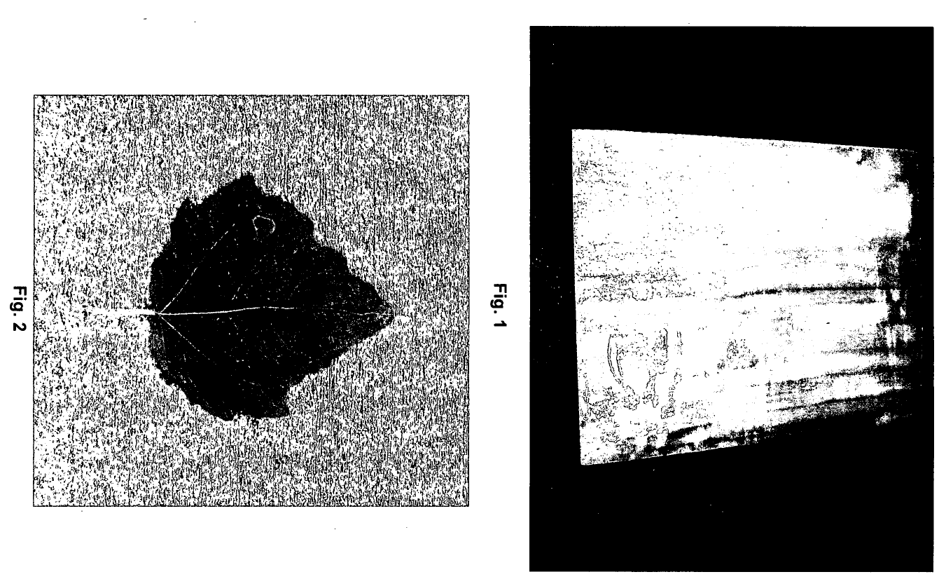

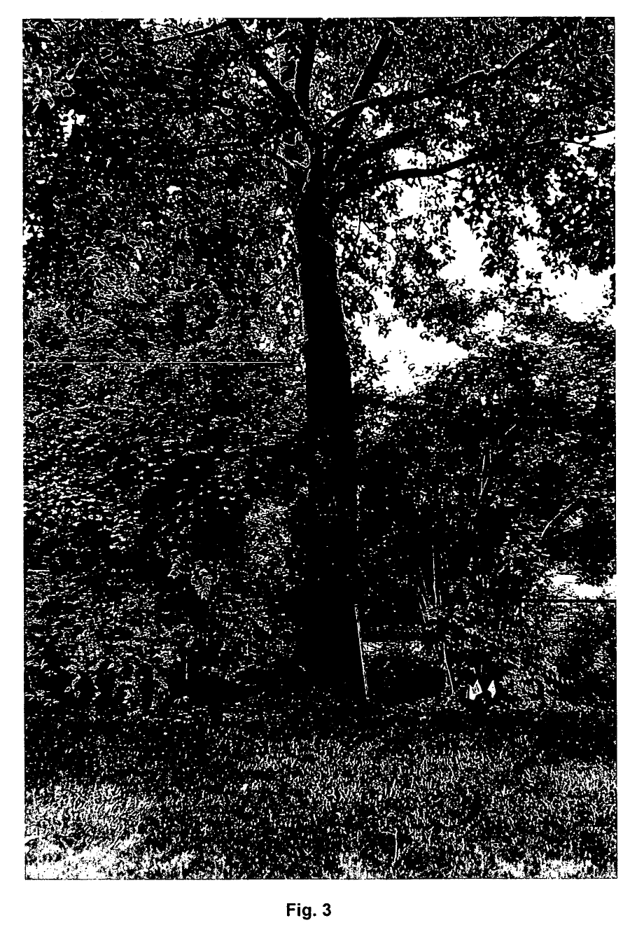

### DESCRIPTION OF THE NEW CULTIVAR

The following detailed description sets forth the distinctive characteristics of ‘Curly Poplar’. The data which define these characteristics were collected from a 23-year-old tree in Evanston, Ill. Color references are to the Munsell Color Charts for Plant Tissues.

### DETAILED BOTANICAL DESCRIPTION

Classification:
   Botanical name.—Populus canescans×Populus alba hybrid.
   Common name.—Poplar.
Trunk:
   Size, at 23 years of age.—Average diameter: 23 inches, measured at 4.5 feet above the ground Height: 68.9 feet.
   Growth habit.—Erect excurrent branching.
   Growth rate.—3 feet/year.
Branches:
   Size.—Crown diameter of tree 23.0 feet at 23 years. Branches aged 15 years: Length: 12 feet Diameter: 5 inches.
   Branching habit.—Dominant main stem with ascending excurrent branching.
Lenticels:
   Size.—⅜ inch.
   Color.—10R 6/8.
   Quantity.—Numerous.
Leaves:
   Immature.—Length: 3-4 inches Width: 2.5-4 inches Shape: Sub-orbicular to deltoid, 3 poorly defined lobes Margin: Irregularly sharply dentate Texture: Loose.
   Mature.—Length: 2.5-3.5 inches Width: 2-2.5 inches Shape: Ovate Margin: Crenate-dentate Texture: Firm Color: Upper surface: 2.5G 5/8 Lower surface: 2.5G 8/4.
   Pubescence distribution.—Lower surface.
   Pubescence color.—Light gray.
   Petiole length.—1.75-2.5 inches.
   Petiole diameter.— 1/16 inch.
   Venation.—Pinnate.
Leaf buds:
   Shape.—Arrow-ovate.
   Length.—⅜-½ inch.
   Width.—⅛- 3/16 inch.
   Pubescence.—Light.
   Pubescence color.—Gray.
   Bud scales.—Size: ¼ inch Shape: Ovate Number: 8.
Flower buds:
   Shape.—Ovate-globose.
   Length.—⅜-½ inch.
   Width.—¼ inch.
   Texture.—Smooth.
Flowers:
   Type.—Dioecious male.
   Time of flowering.—Early — mid-April.
   Length of flowering period.—1 week.
   Size.—Length: 2-4 inches Diameter: ⅜-½ inch.
Disease and insect resistance: Resistant to anaerobic bacterial infection. No known insect pests.

### COMPARISON WITH MOST SIMILAR CULTIVAR

‘Curly Poplar’is a distinct variety of poplar due to its highly figured wood. The highly figured wood is due to the tree's undulating cross grain which results in a strong mottle-figured grain. All the other characteristics of this poplar tree are similar to those of Populus canescans and Populus alba.

We claim:

1. A new and distinct poplar tree as shown and described herein.
